(19) United States
(12) Patent Application Publication
(10) Pub. No.: US 2019/0328671 A1
(43) Pub. Date: Oct. 31, 2019

(54) PARTICLES, PHARMACEUTICAL PREPARATION, TOPICAL DRUG, AND COSMETIC PRODUCT

(71) Applicant: SEKISUI CHEMICAL CO., LTD., Osaka (JP)

(72) Inventors: Saori Tone, Osaka (JP); Izumi Matsumoto, Osaka (JP); Takayuki Akamine, Osaka (JP); Daichi Kawamura, Osaka (JP); Kazushi Itou, Osaka (JP); Yoshiko Abe, Tokyo (JP)

(21) Appl. No.: 16/508,273

(22) Filed: Jul. 10, 2019

Related U.S. Application Data
(62) Division of application No. 15/773,982, filed on May 4, 2018.

(30) Foreign Application Priority Data
Dec. 25, 2015 (JP) 2015-253458

(51) Int. Cl. A61K 9/19; A61K 9/70; A61K 47/26; A61K 9/00; A61K 8/02; A61K 47/32; A61Q 19/00; A61K 8/73; A61K 31/13; A61K 47/36; A61K 8/81

(52) U.S. Cl. CPC A61K 9/19; A61K 9/7053; A61K 47/26; A61K 9/0014; A61K 8/0241; A61K 8/8152; A61Q 19/00; A61K 8/735; A61K 31/13; A61K 47/36; A61K 47/32

(57) ABSTRACT

Provided are an active ingredient-containing particle with improved shape retainability. The particle includes a first fraction containing an active ingredient, a second fraction containing a surfactant, and at least one water-soluble polymer selected from the group consisting of a polysaccharide and a polymer having 2-methacryloyloxyethyl phosphorylcholine as a constituent unit.

### CROSS-REFERENCE TO RELATED APPLICATIONS

This application is a Divisional Application of patent application Ser. No. 15/773,982, filed on May 4, 2018, which is a 371 application of Application Serial No. PCT/JP2016/088451, filed on Dec. 22, 2016, which is based on Japanese Patent Application No. 2015-253458, filed on Dec. 25, 2015, the entire contents of which are hereby incorporated by reference.

### TECHNICAL FIELD

The present invention relates to active ingredient-containing a particle, and a formulation, an external preparation and a cosmetic product that contain the same.

### BACKGROUND ART

In order to improve the permeability of an active ingredient into the body, various techniques have been proposed. For example, in Patent Literature 1 to 3, formulations using a particle formed of assemblies of a hydrophilic active ingredient-containing phase and a surfactant-containing phase (active ingredient-containing particle), i.e., so-called S/O (Solid in Oil) formulations, are disclosed. In the technique, a surfactant allows the permeability of an active ingredient into the body to be improved and the in vivo kinetics of the active ingredient to be controlled.

### CITATION LIST

### Patent Literature

Patent Literature 1: Japanese Patent No. 4349639
Patent Literature 2: Japanese Patent No. 4843494
Patent Literature 3: International Publication No. WO 2005/094789

### SUMMARY OF INVENTION

### Technical Problem

The active ingredient-containing particle, however, has a problem that although the shape can be retained immediately after manufacturing, the shape changes over time, so that the active ingredient leaks out from the particle. The leakage of the active ingredient causes crystallization of the active ingredient, which reduces the permeability into the body. The Problem becomes further evident under higher temperature conditions such as heating conditions in a formulation process.

Meanwhile, in order to improve the shape retainability of the active ingredient-containing particle, blending a protein such as BSA as a stabilizer into the particle (e.g., Patent Literature 3) has been proposed. A protein, however, is denatured by heat, so that the method is unsuitable considering the problem.

Also, the present inventors found that when a protein such as BSA is used as a stabilizer, the shape of a particle changes in an early stage under relatively high temperature. In order to improve the practicability of the active ingredient-containing particle, further improvement in the shape retainability of the particle is therefore required.

In view of the above, an object of the present invention is to provide an active ingredient-containing particle with improved shape retainability.

### Solution to Problem

Through extensive studies to solve the above problem, the present inventors found that a particle comprising a first fraction containing an active ingredient, a second fraction containing a surfactant, and at least one water-soluble polymer selected from the group consisting of a polysaccharide and a polymer having 2-methacryloyloxyethyl phosphorylcholine as a constituent unit, can solve the problem. The present invention has been accomplished through further trials and errors based on the finding, including the following aspects.

Aspect 1: A particle comprising a first fraction containing an active ingredient, a second fraction containing a surfactant, and at least one water-soluble polymer selected from the group consisting of a polysaccharide and a polymer having 2-methacryloyloxyethyl phosphorylcholine as a constituent unit.

Aspect 2: The particle according to aspect 1, wherein the particle includes a part or the whole of the surface of the first fraction directly or indirectly covered with the second fraction.

Aspect 3: The particle according to aspect 1 or 2, wherein the first fraction contains the water-soluble polymer.

Aspect 4: The particle according to any of aspects 1 to 3, having a water content of 20 wt % or less.

Aspect 5: The particle according to any of aspects 1 to 4, wherein the water-soluble polymer has a molecular weight of 2000 or more.

Aspect 6: The particle according to any of aspects 1 to 5, wherein the water-soluble polymer has a molecular weight of 50000 or more.

Aspect 7: The particle according to any of aspects 1 to 6, wherein the polysaccharide is at least one selected from the group consisting of hyaluronic acid and salts thereof.

Aspect 8: The particle according to any of aspects 1 to 6, wherein the polymer having 2-methacryloyloxyethyl phosphorylcholine as a constituent unit is a copolymer of 2-methacryloyloxyethyl phosphorylcholine with a hydrophobic monomer.

Aspect 9: The particle according to any of aspects 1 to 8, wherein a weight ratio between the active ingredient and the water-soluble polymer is 1:0.02 to 1:5.

Aspect 10: A formulation comprising the particle according to any of aspects 1 to 9.

Aspect 11: The formulation according to aspect 10, having a water content of 20 wt % or less.

Aspect 12: The formulation according to aspect 10 or 11, wherein a weight ratio between the active ingredient and the surfactant is 1:3 to 1:50.

Aspect 13: An external preparation comprising the formulation according to any of aspects 10 to 12.

Aspect 14: A cosmetic product comprising the formulation according to any of aspects 10 to 12.

### Advantageous Effect of Invention

According to the present invention, an active ingredient-containing particle with further improved shape retainability due to a specific water-soluble polymer can be provided. The particle of the present invention has excellent shape retainability, so that the storage stability of a product can be achieved. With use of a polymer having 2-methacryloyloxyethyl phosphorylcholine as a constituent unit as the specific water-soluble polymer, even higher transdermal absorption can be also achieved.

Also, the water-soluble polymer for use as a stabilizer is stable even under high temperature conditions in a formulation process of the particle and the like.

### BRIEF DESCRIPTION OF DRAWINGS

FIG. 1 is a schematic view of a cell for use in testing the skin permeability of a drug in Test Example 2.

FIG. 2 is observed images illustrating the results in Test Example 3.

### DESCRIPTION OF EMBODIMENTS

In the present specification, the expression “containing” includes the concepts of “comprising” and “essentially composed of”.

1. Particle

The present invention relates to a particle (also referred to as “a particle of the present invention” in the present specification) comprising a first fraction containing an active ingredient, a second fraction containing a surfactant, and at least one water-soluble polymer (also referred to as “water-soluble polymer of the present invention” in the present specification) selected from the group consisting of a polysaccharide and a polymer having 2-methacryloyloxyethyl phosphorylcholine as a constituent unit. The particle is described as follows.

The particle of the present invention comprises at least two fractions, i.e., a first fraction containing an active ingredient and a second fraction containing a surfactant, and a water-soluble polymer of the present invention.

The first fraction and the second fraction have only to be connected to each other (preferably through intermolecular force) to form an assembly. In the particle of the present invention, from the viewpoints of absorption into the body and sustained release of the active ingredient, it is preferable that a part or the whole of the surface of the first fraction (for example, 30% or more, preferably 50% or more, more preferably 70% or more, still more preferably 85% or more, further more preferably 95% or more, particularly preferably 99% or more, of the surface of the first fraction) is directly or indirectly (preferably directly) covered with the second fraction. Examples of the embodiment of the particle include a core-shell structure having a first fraction as a core part and a second fraction as a shell part enwrapping the core part.

In the particle of the present invention, the water-soluble polymer of the present invention may be included in any one of the first fraction, the second fraction, and a fraction (a third fraction) that lies between the first fraction and the second fraction. Although a limited interpretation is not desired, the particle of the present invention is typically obtained by drying a W/O emulsion of a water phase including an active ingredient and an oil phase including a surfactant, and the water-soluble polymer of the present invention is presumed to be present together with the active ingredient in the water phase in the manufacturing process. It is therefore presumed that in the particle of the present invention, the water-soluble polymer of the present invention is present in the first fraction. In that case, the distribution state of the water-soluble polymer of the present invention in the first fraction is not particularly limited, and examples thereof include a state with the polymer distributed in the surface layer of the first fraction to cover the active ingredient in the inner layer, and a state with the polymer mixed with the active ingredient.

The number average particle diameter of the particle of the present invention is not particularly limited. The number average particle diameter is, for example, 1 nm to 800 nm, preferably 1 nm to 500 nm, more preferably 1 nm to 100 nm.

The shape of the particle is not particularly limited. The shape of the particle may be, for example, spherical, rod-like, and spheroid.

In the present invention, the number average particle diameter of the particle is calculated in dynamic light scattering when the particle is dispersed in a solvent (e.g., squalane).

The moisture content of the particle is preferably 20 wt % or less, more preferably 10 wt % or less, still more preferably 5 wt % or less, further more preferably 1 wt % or less, particularly preferably substantially free of water. In other words, the particle of the present invention is different from a particle in a W/O emulsion.

In the present invention, it is preferable that the first fraction is a solid. In this case, the stability in a base to be described below is further improved. Accordingly, dispersing the particle in a base phase as oil phase allows a formulation having an S/O (solid in oil) structure to be formed.

1.1 Water-Soluble Polymer

The water-soluble polymer of the present invention is at least one selected from the group consisting of a polysaccharide and a polymer having 2-methacryloyloxyethyl phosphorylcholine as a constituent unit. In other words, the water-soluble polymer of the present invention is at least one polymer between a polysaccharide and a polymer having 2-methacryloyloxyethyl phosphorylcholine as a constituent unit.

Although a limited interpretation is not desired, the mechanism for the water-soluble polymer of the present invention to improve the shape retainability of the particle is presumed as follows. The water-soluble polymer of the present invention alone is usually folded without particle formation in a solution, capable of being in a flexible state. Meanwhile, it is presumed that the water-soluble polymer of the present invention is present together with an active ingredient in the water phase in the manufacturing process of the particle of the present invention as described above. It is presumed that the water-soluble polymer of the present invention is present there capturing or covering the active ingredient due to having the properties (properties capable of being present in a flexible state). It is therefore presumed that also in the particle of the present invention eventually obtained, the water-soluble polymer of the present invention is present capturing the active ingredient or covering the first fraction including the active ingredient. It is presumed that in the particle of the present invention, the active ingredient is, therefore, more firmly retained, so that the high shape retainability can be achieved.

The polysaccharide is not particularly limited, and examples thereof include mucopolysaccharides such as (in particular, acidic mucopolysaccharides)hyaluronic acid, chondroitin, chondroitin sulfate, dermatan sulfate, and kerato sulfate, and salts thereof. Among them, from the viewpoint of more reliably exhibiting the effect of the present invention, hyaluronic acid, chondroitin, chondroitin sulfate, and salts thereof are preferred, hyaluronic acid, chondroitin, and salts thereof are more preferred, and hyaluronic acid and salts thereof are still more preferred.

The salt of polysaccharide is not particularly limited as long as the salt can be formed from a polysaccharide, and examples thereof include an alkaline metal salt such as a sodium salt and a potassium salt, and an alkaline earth metal salt such as a calcium salt and a magnesium salt. An alkaline metal salt is preferred and a sodium salt is more preferred.

The polysaccharide can be used singly or in any combination of two or more.

The polymer having 2-methacryloyloxyethyl phosphorylcholine as a constituent unit is not particularly limited as long as the constituent monomer has 2-methacryloyloxyethyl phosphorylcholine. In the present specification, these are collectively referred to as “phospholipid-like water-soluble polymer” in some cases.

As the phospholipid-like water-soluble polymer, 2-methacryloyloxyethyl phosphorylcholine homopolymer or a copolymer of 2-methacryloyloxyethyl phosphorylcholine and a hydrophobic monomer is preferred. The 2-methacryloyloxyethyl phosphorylcholine homopolymer is not particularly limited as long as the constituent monomer is made only of 2-methacryloyloxyethyl phosphorylcholine.

The hydrophobic monomer is not particularly limited as long as the copolymer eventually obtained is pharmaceutically, pharmacologically (in drug making) or physiologically acceptable, and preferred examples include a monomer represented by a general formula (A): CH₂═C(—R1)-COO—R2 (also referred to as “monomer (A)” in the present specification in some cases).

In the general formula (A), R1 represents a hydrogen atom or a methyl group, preferably a methyl group.

Alternatively, in the general formula (A), R1 represents a hydrogen atom, or an alkyl group having 1 to 6 carbon atoms. Examples of the alkyl group include a methyl group, an ethyl group, an n-propyl group, an n-butyl group, an n-pentyl group, and an n-hexyl group. Examples of R2 include an alkyl group preferably having 1 to 5 carbon atoms, an alkyl group more preferably having 1 to 4 carbon atoms, and an alkyl group particularly preferably having 4 carbon atoms (n-butyl group).

Suitable examples of the monomer (A) include butyl methacrylate (BMA with a methyl group R1 and an n-butyl group R2), methyl methacrylate (MMA with a methyl group R1 and a methyl group R2), 2-hydroxyethyl methacrylate (HEMA with a methyl group R1 and a hydroxyethyl group R2); 2-hydroxyethyl methacrylate and butyl methacrylate are more preferred; and butyl methacrylate is particularly preferred.

When the monomer (A) can take the form of a salt (e.g., with a hydrogen atom R1), the monomer (A) may be a salt. Examples of the monomer (A) in the form of a salt include the salt of an alkaline metal such as sodium and potassium.

The component ratio between 2-methacryloyloxyethyl phosphorylcholine and a hydrophobic monomer differs depending on the structure of the monomer for use and the like, for example, being usually 5 to 50 mol %, preferably 10 to 40 mol %, still more preferably 15 to 25 mol %, of hydrophobic monomer relative to the copolymer.

The phospholipid-like water-soluble polymer for use may be manufactured in accordance with or according to a known synthetic method, or may be a commercially available product such as LIPIDURE Series manufactured by NOF Corporation.

The phospholipid-like water-soluble polymer may be used alone or in any combination of two or more.

As the water-soluble polymer in the present invention, use of the phospholipid-like water-soluble polymers as described above is preferred. In that case, the shape retainability can be further improved while the transdermal absorption is further enhanced.

The molecular weight of the water-soluble polymer of the present invention is not limited as long as the polymer is pharmaceutically, pharmacologically (in drug making) or physiologically acceptable. The lower limit of the molecular weight is desirably high from the viewpoint of further improving the shape retainability of the particle of the present invention, and can be, for example, 1000, preferably 2000, more preferably 10000, still more preferably 50000, further more preferably 200000, further more preferably 500000, further more preferably 1000000. On the other hand, the upper limit of the molecular weight is not particularly limited as long as the particle of the present invention can be formed. The upper limit is, for example, 8000000, preferably 5000000.

1.2 First Fraction

The first fraction comprises at least an active ingredient.

The active ingredient is not particularly limited as long as the component has a physiological activity. Preferably, the component is blended to exert its physiological activity. In this preferred embodiment, any component having a physiological activity that is not blended to exert the physiological activity from the view points of the amount blended, the blending method, etc., is not included in the active ingredient. Examples of the active ingredient include components that are blended as active ingredient into pharmaceuticals, cosmetics, etc.

As the active ingredient to be blended into pharmaceuticals, any of those for systemic action and those for local action can be used.

Specific examples of the active ingredient blended into pharmaceuticals include, but are not particularly limited thereto, therapeutic agents for dementia, antiepileptics, antidepressants, anti-Parkinson's drugs, anti-allergic drugs, anti-cancer agents, antidiabetic agents, antihypertensive agents, therapeutic agents for ED, dermatologic agents, local anesthetics, and pharmaceutically acceptable salts thereof. More specifically examples include memantine, donepezil, rivastigmine, galantamine, nitroglycerin, lidocaine, fentanyl, male hormones, female hormones, nicotine, clomipramine, diphenhydramine, nalfurafine, metoprolol, fesoterodine, vardenafil, tandospirone, beraprost sodium, taltirelin, lurasidone, nefazodone, rifaximin, benidipine, doxazosin, nicardipine, formoterol, lomerizine, amlodipine, vardenafil, octreotide, teriparatide, bucladesine, cromoglicic acid, and pharmaceutically acceptable salts thereof.

The pharmaceutically acceptable salts are not particularly limited, and any of acidic salts and basic salts can be employed. Examples of the acidic salts include inorganic acidic salts such as hydrochlorides, hydrobromides, sulfates, nitrates, and phosphate, and organic acidic salts such as acetates, propionates, tartrates, fumarates, maleates, malates, citrates, methanesulfonates, benzenesulfonates, and p-toluenesulfonates. Examples of the basic salts include salts of alkali metals such as sodium salts and potassium salts, and alkaline earth metal salts such as calcium salts and magnesium salts. Examples of the salt of the active ingredient include memantine hydrochloride, donepezil hydrochloride, rivastigmine tartrate, galantamine hydrobromide, clomipramine hydrochloride, diphenhydramine hydrochloride, nalfurafine hydrochloride, metoprolol tartrate, fesoterodine fumarate, vardenafil hydrochloride hydrate, tandospirone citrate, beraprost sodium, lurasidone hydrochloride, nefazodone hydrochloride, benidipine hydrochloride, doxazosin mesylate, nicardipine hydrochloride, formoterol fumarate, lomerizine hydrochloride, amlodipine besylate, vardenafil hydrochloride, octreotide acetate, teriparatide acetate, bucladesine sodium, sodium cromoglycate.

The active ingredient to be blended into cosmetics is not particularly limited as long as skin permeation is required, and examples thereof include vitamin components such as vitamin C and Vitamin E, moisturizing components such as hyaluronic acid, ceramide, and collagen, whitening components such as tranexamic acid and arbutin, hair growth components such as minoxidil, cosmetic components such as FGF (fibroblast growth factor), EGF (epidermal growth factor), and the salts and derivatives thereof.

Preferably, the active ingredient is hydrophilic.

When an active ingredient is hydrophilic, though not particularly limited thereto, the active ingredient typically has the properties below: a molecular weight of 10000 or less, and an octanol/water partition coefficient of −6 to 6

In the above description, the molecular weight is preferably 1000 or less. The lower limit of molecular weight is typically 50 or more, though not particularly limited.

In the above description, the octanol/water partition coefficient is preferably −3 to 5, more preferably −1 to 4.

In the present invention, the octanol/water partition coefficient is obtained as follows. After the chemical is added into a flask containing octanol and an aqueous buffer at pH 7, the mixture is shaken. Based on the chemical concentration in each phase, the octanol/water partition coefficient is calculated by the following equation.

Octanol/water partition coefficient=Log 10(concentration in octanol phase/concentration in aqueous phase)

The amount of the active ingredient contained in the particle of the present invention depends on the type of the active ingredient, and the weight of raw material added may be, for example, 0.1 to 30 wt % (based on the total weight of all the raw materials contained in the particle).

The active ingredient can be used alone or in any combination of two or more.

The first fraction may further contain at least one other component in addition to the active ingredient. Examples of the other component include absorption enhancer, irritation reducing agents and antiseptics, though not limited thereto.

Specific examples of the absorption enhancer include higher alcohols, N-acyl sarcosine and salts thereof, higher monocarboxylic acids, higher monocarboxylic acid esters, aromatic monoterpene fatty acid esters, dicarboxylic acids having 2 to 10 carbon atoms and salts thereof, polyoxyethylene alkyl ether phosphate and salts thereof, lactic acid, lactates, and citric acid, though not particularly limited thereto. One or two or more absorption enhancers may be contained. The absorption enhancer content in the first fraction may be appropriately set depending on the type, and the weight ratio between the active ingredient and the absorption enhancer in the formulation may be set, for example, at 1:0.01 to 1:50.

Specific examples of the irritation reducing agent include hydroquinone glycosides, pantethine, tranexamic acid, lecithin, titanium oxide, aluminum hydroxide, sodium nitrite, sodium hydrogen nitrite, soy lecithin, methionine, glycyrrhetinic acid, BHT, BHA, vitamin E and derivatives thereof, vitamin C and derivatives thereof, benzotriazole, propyl gallate, and mercaptobenzimidazole, though not particularly limited thereto. One or two or more irritation reducing agents may be contained. The irritation reducing agent content in the first fraction may be appropriately set depending on the type, and may be set, for example, at 0.1 wt % to 50 wt % in the formulation.

Specific examples of the antiseptic include methyl parahydroxybenzoate, propyl parahydroxybenzoate, phenoxyethanol and thymol, though not particularly limited thereto. The antiseptic content in the first fraction may be appropriately set depending on the type, and may be set, for example, at 0.01 wt % to 10 wt % in the formulation. One or two or more antiseptics may be contained.

1.3 Second Fraction

The second fraction comprises at least a surfactant.

A surfactant having a weight average HLB (abbreviation of Hydrophile Lypophile Balance) value of 10 or less, preferably 5 or less, more preferably 3 or less, may be used.

The HLB value in the present invention is an indicator of whether an emulsifier is hydrophilic or lipophilic, taking a value of 0 to 20. The smaller HLB value indicates higher lipophilic. The HLB value is calculated from the following Griffin equation in the present invention.

HLB value=20×{(molecular weight of hydrophilic moiety)/(total molecular weight)}

The weight average HLB value is calculated as follows.

For example, when surfactant materials have HLB values A, B, and C, with weights of x, y, and z, respectively, the calculated weight average HLB value is: (xA+yB+zC)/(x+y+z).

The surfactant has a melting point of preferably 50° C. or lower, more preferably 40° C. or lower, from the viewpoint of permeability.

The surfactant is not particularly limited, and may be appropriately selected depending on the application. For example, it may be selected widely from those that can be used in pharmaceuticals and cosmetics. A plurality of surfactants may be used in combination.

The surfactant may be any of nonionic surfactants, anionic surfactants, cationic surfactants and amphoteric surfactants.

Examples of the nonionic surfactant include fatty acid esters, fatty alcohol ethoxylates, polyoxyethylene alkyl phenyl ethers, alkyl glycosides and fatty acid alkanolamides, and polyoxyethylene castor oil and hardened castor oil, though not particularly limited thereto.

The fatty acid ester is not particularly limited, and sugar fatty acid esters are preferred. Specific examples include esters of a fatty acid such as erucic acid, oleic acid, lauric acid, stearic acid and behenic acid, and sucrose.

Examples of the other fatty acid ester include esters of at least one of glycerin, polyglycerin, polyoxyethylene glycerin, sorbitan and polyoxyethylene sorbitol, and a fatty acid, though not particularly limited.

Examples of the anionic surfactant include alkyl sulfates, polyoxyethylene alkyl ether sulfate, alkyl benzene sulfonates, fatty acid salts and phosphates.

Examples of the cationic surfactant include alkyltrimethylammonium salts, dialkyldimethylammoniumm salts, alkyldimethylbenzylammonium salts and amine salts.

Examples of the amphoteric surfactant include alkylamino fatty acid salts, alkyl betaines and alkyl amine oxides.

As the surfactant, sucrose fatty acid esters, glycerin fatty acid esters, polyoxyethylene glycerin fatty acid esters, sorbitan fatty acid esters, polyoxyethylene sorbit fatty acid esters, polyoxyethylene castor oil and hardened castor oil are particularly preferably used.

The surfactants may include, but are not particularly limited to, those having a hydrocarbon chain (alkyl chain, alkenyl chain, alkynyl chain, etc.). The hydrocarbon chain length is not particularly limited, and may be selected widely from those having 8 to 30 carbon atoms in the main chain. The particularly preferable length is 10 to 24.

In the case using only a surfactant having a hydrocarbon chain, or in the case using a surfactant having a hydrocarbon chain in combination with other surfactants, the particle of the present invention has excellent absorption sustainability when having a weight ratio between the active ingredient and the total hydrocarbon chains contained in the surfactants of 1:1 to 1:70. In this regard, the weight ratio is preferably 1:2 to 1:70 or 1:2 to 1:50, more preferably 1:3 to 1:30, further more preferably 1:5 to 1:20.

The surfactant can be used alone or in any combination of two or more.

The second fraction may further contain at least one other component in addition to the surfactant. Examples of the other component may include, but are not particularly limited to, irritation reducing agents, analgesics, absorption enhancers, stabilizers, and antiseptics.

Specific examples of the irritation reducing agent may include, but are not particularly limited to, hydroquinone glycosides, pantethine, tranexamic acid, lecithin, titanium oxide, aluminum hydroxide, sodium nitrite, sodium hydrogen nitrite, soy lecithin, methionine, glycyrrhetinic acid, BHT, BHA, vitamin E and derivatives thereof, vitamin C and derivatives thereof, benzotriazole, propyl gallate, and mercptobenzimidazole. One or two or more irritation reducing agents may be contained. The content of irritation reducing agents in the second fraction may be appropriately set depending on the type, and may be set at, for example, 0.1 wt % to 50 wt % in the formulation.

Specific examples of the analgesic may include, but are not particularly limited to, a local anesthetic such as procaine, tetracaine, lidocaine, dibucaine and prilocaine, and salts thereof. One or two or more analgesics may be contained. The content of analgesic in the second fraction may be appropriately set depending on the type, and may be set at, for example, 0.1 wt % to 30 wt % in the formulation.

Specific examples of the absorption enhancer may include, but are not particularly limited to, higher alcohols N-acyl sarcosine and salts thereof, higher monocarboxylic acids, higher monocarboxylic acid esters, aromatic monoterpene fatty acid esters, dicarboxylic acids having 2 to 10 carbon atoms and salts thereof, polyoxyethylene alkyl ether phosphates and salts thereof, lactic acid, lactates and citric acid. One or two or more absorption enhancers may be contained. The content of absorption enhancers in the second fraction may be appropriately set depending on the type, and may be set, for example, at 0.1 wt % to 30 wt % in the formulation.

Stabilizers have a function for stabilizing the core-shell structure, preventing the unintentional collapse of the core-shell structure in an early stage, and ensuring the sustained release effect of the active ingredient.

Specific examples of the stabilizer may include, but are not particularly limited to, fatty acids and salts thereof, p-hydroxybenzoic acid esters such as methyl paraben and propyl paraben, alcohols such as chlorobutanol, benzyl alcohol, and phenylethyl alcohol, thimerosal, acetic anhydride, sorbic acid, sodium hydrogen sulfite, L-ascorbic acid, sodium ascorbate, butylhydroxyanisole, butylhydroxy toluene, propyl gallate, tocopherol acetate, dl-α-tocopherol and polysaccharides. One or two or more stabilizers may be contained. The content of stabilizers in the second fraction may be appropriately set depending on the type, and the weight ratio between sucrose fatty acid ester and stabilizers in the formulation may be set, for example, at 1:0.01 to 1:50.

Specific examples of the antiseptic may include, but are not particularly limited to, methyl parahydroxybenzoate, propyl parahydroxybenzoate, phenoxyethanol and thymol. One or two or more antiseptics may be contained. The content of antiseptics in the second fraction may be appropriately set depending on the type, and may be set, for example, at 0.01 wt % to 10 wt % in the formulation.

2. Formulation

The formulation of the present invention contains at least the particle of the present invention.

The content of the particle of the present invention in the formulation of the present invention is, preferably 35 wt % or more, more preferably 45 wt % or more, though not particularly limited thereto.

The weight ratio between the active ingredient and the surfactant (active ingredient weight/surfactant weight) in the formulation of the present invention may be appropriately set in a range where the effect of the present invention can be attained, for example, 1:2 to 1:100. In that case, the formulation of the present invention is excellent in absorption into the body. In this regard, the weight ratio is controlled at preferably 1:3 to 1:50, more preferably 1:3 to 1:30.

In the formulation of the present invention, the weight ratio between the active ingredient and the water-soluble polymer of the present invention (weight of active ingredien:weight of water-soluble polymer of the present invention) can be appropriately set within a range that the effect of the present invention is achieved. From the viewpoint of enhancing the shape retainability of the particle of the present invention, the ratio is, for example, 1:0.01 to 1:10, preferably 1:0.02 to 1:5, more preferably 1:0.05 to 1:2, still more preferably 1:0.08 to 1:1.5.

Depending on the type of active ingredient, the formulation of the present invention may be used in varieties of applications including external pharmaceuticals such as skin external preparations, eye drops, nasal drops, suppositories and oral preparations, and cosmetics.

The formulation of the present invention provides a typical sustained release of 1 day to 1 week, though not particularly limited thereto. In a preferred embodiment, the formulation is applied once per day to once per week.

The target disease of the formulation of the present invention for use as external pharmaceuticals differs depending on the type of active ingredient.

The formulation of the present invention is not particularly limited, and can be used as patches (plasters, tapes (reservoir-type, matrix-type, etc.) such as emplastrums, cataplasms, transdermal patches, microneedles, etc.), ointments, liquids for external use (liniments, lotions, etc.), sprays (aerosols for external use, pump sprays, etc.), creams, gels, eye drops, eye ointments, nasal drops, suppositories, semisolids for application to rectum, and enema agents.

The formulation of the present invention has a water content of preferably 20 wt % or less, more preferably substantially zero. The shape retainability of the particle of the present invention can be thereby further enhanced. Along with the shape retainability inherent to the particle, the leakage of the active ingredient from the particle and the consequent crystallization of the active ingredient can be further suppressed, so that higher absorption into the body can be achieved. From this viewpoint, the formulation of the present invention is preferably used as an agent with a water content controlled at 20 wt % or less (more preferably as an agent with substantially no water content), such as tapes, transdermal patches, ointments, gels, eye drops, and eye ointments.

2. 1 Base Phase

The formulation of the present invention may further contain a base-containing phase (base phase), which contains the particle of the present invention. In that case, the particle is dispersed or dissolved in the base phase.

The base is not particularly limited and may be widely selected from those that can be used as pharmaceuticals (external pharmaceuticals, in particular) and cosmetics.

As described above, preferably the particle of the present invention include a first fraction of solid. In the case of a base phase of oil, such particle is dispersed in the base phase as oil phase, so that an S/O (solid in oil) formulation can be formed. The S/O formulation can be obtained, for example, by dispersing the particle obtained by a production method including the step of drying W/O emulsion described below in an oil phase.

The base may be appropriately selected from those suitable for dispersing or dissolving the particle corresponding to the intended use, and is not particularly limited.

A plurality of bases may be used in combination.

Examples of the base include, but are not particularly limited to, oily bases and aqueous bases. Examples of the oily base include elastomers, vegetable oils, animal oils, neutral lipids, synthetic oils, sterol derivatives, waxes, hydrocarbons, monoalcohol carboxylic acid esters, oxy acid esters, polyhydric alcohol fatty acid esters, silicones, higher alcohols, higher fatty acids, and fluorine oils. Examples of the aqueous base include water and (poly)alcohols.

The elastomer is not particularly limited, and examples thereof include rubbers such as a styrene-isoprene-styrene block copolymer (SIS), a styrene-butadiene-styrene block copolymer (SBS), a styrene-ethylene-butylene-styrene block copolymer (SEBS), polyisobutylene (PIB) and isoprene rubber (IR), silicones such as silicone rubber, urethanes, and acrylics.

Examples of the vegetable oils include, but are not particularly limited to, soybean oil, sesame oil, olive oil, coconut oil, palm oil, rice oil, cottonseed oil, sunflower oil, rice bran oil, cacao butter, corn oil, safflower oil, castor oil and rapeseed oil.

Examples of the animal oil include, but are not particularly limited to, mink oil, turtle oil, fish oil, beef oil, horse oil, lard, and shark squalane.

Examples of the neutral lipid include, but are not particularly limited to, triolein, trilinolein, trimyristin, tristearin and triarachidonin.

Examples of the synthetic oil include, but are not particularly limited to, phospholipids and azone.

Examples of the sterol derivative include, but are not particularly limited to, dihydrocholesterol, lanosterol, dihydrolanosterol, phytosterol, and cholic acid and cholesteryl linoleate.

Examples of the wax include candelilla wax, carnauba wax, rice wax, Japan wax, beeswax, montan wax, ozokerite, ceresin, paraffin wax, microcrystalline wax, petrolatum, Fischer-Tropsch wax, polyethylene wax and ethylene-propylene copolymers.

Examples of the hydrocarbon include liquid paraffin (mineral oil), heavy liquid isoparaffin, light liquid isoparaffin, α-olefin oligomers, polyisobutene, hydrogenated polyisobutene, polybutene, squalane, olive-derived squalane, squalene, vaseline, and solid paraffin.

Examples of the monoalcohol carboxylic acid esters include octyldodecyl myristate, hexyldecyl myristate, octyldodecyl isostearate, cetyl palmitate, octyldodecyl palmitate, cetyl octanoate, hexyldecyl octanoate, isotridecyl isononanoate, isononyl isononanoate, octyl isononanoate, isodecyl isononanoate, isodecyl neopentanoate, isotridecyl neopentanoate, isostearyl neopentanoate, octyldodecyl neodecanoate, oleyl oleate, octyldodecyl oleate, octyldodecyl ricinoleate, octyldodecyl lanolate, hexyldecyl dimethyloctanoate, octyldodecyl erucate, hydrogenated castor oil isostearate, ethyl oleate, avocado oil fatty acid ethyl, isopropyl myristate, isopropyl palmitate, octyl palmitate, isopropyl isostearate, isopropyl lanolate, diethyl sebacate, diisopropyl sebacate, dioctyl sebacate, diisopropyl adipate, dibutyloctyl sebacate, diisobutyl adipate, dioctyl succinate, and triethyl citrate.

Examples of the oxy acid esters include cetyl lactate, diisostearyl malate, and hydrogenated castor oil monoisostearate.

Examples of the poly alcohol fatty acid esters include glyceryl trioctanoate, glyceryl trioleate, glyceryl triisostearate, glyceryl diisostearate, glyceryl tri(caprylate/caprate), glyceryl tri(caprylate/caprate/myristate/stearate), hydrogenated rosin triglyceride (hydrogenated ester gum), rosin triglyceride (ester gum), glyceryl(behenate/eicosadioate), trimethylolpropane trioctanoate, trimethylolpropane triisostearate, neopentyl glycol dioctanoate, neopentyl glycol dicaprate, 2-butyl-2-ethyl-1,3-propanediol dioctanoate, propylene glycol dioleate, pentaerythrityl tetraoctanoate, hydrogenated rosin pentaerythrityl, ditrimethylolpropane triethylhexanoate, ditrimethylolpropane(isostearate/sebacate), pentaerythrityl triethylhexanoate, dipentaerythrityl(hydroxystearate/stearate/rosinate), diglyceryl diisostearate, polyglyceryl tetraisostearate, polyglyceryl-10 nonaisostearate, polyglyceryl-8 deca(erucate/isostearate/ricinoleate), diglyceryl(hexyldecanoate/sebacate) oligoester, glycol distearate (ethylene glycol distearate), 3-methyl-1,5-pentanediol dineopentanoate, and 2,4-diethyl-1,5-pentadiol dineopentanoate.

Examples of the silicones include dimethicone (dimethyl polysiloxane), highly polymerized dimethicone (highly polymerized dimethylpolysiloxane), cyclomethicone (cyclic dimethylsiloxane, decamethylcyclopentasiloxane), phenyl trimethicone, diphenyl dimethicone, phenyl dimethicone, stearoxy propyl dimethylamine, (aminoethyl aminopropyl methicone/dimethicone) copolymers, dimethiconol, dimethiconol crosspolymers, silicone resins, silicone rubber, amino-modified silicones such as aminopropyl dimethicone and amodimethicone, cation-modified silicones, polyether-modified silicones such as dimethicone copolyol, polyglycerin-modified silicone, sucrose-modified silicones, carboxylic acid-modified silicones, phosphoric acid-modified silicone, sulfuric acid-modified silicones, alkyl-modified silicone, fatty acid-modified silicones, alkyl ether-modified silicones, amino acid-modified silicones, peptide-modified silicones, fluorine-modified silicones, cation-modified or polyether-modified silicones, amino-modified or polyether-modified silicones, alkyl-modified or polyether modified silicones, and polysiloxane/oxyalkylene copolymers.

Examples of the higher alcohols include cetanol, myristyl alcohol, oleyl alcohol, lauryl alcohol, cetostearyl alcohol, stearyl alcohol, arachyl alcohol, behenyl alcohol, jojoba alcohol, chimyl alcohol, selachyl alcohol, batyl alcohol, hexyl decanol, isostearyl alcohol, 2-octyldodecanol and dimer diols.

Examples of the higher fatty acids include lauric acid, myristic acid, palmitic acid, stearic acid, isostearic acid, behenic acid, undecylenic acid, 12-hydroxystearic acid, palmitoleic acid, oleic acid, linoleic acid, linolenic acid, erucic acid, docosahexaenoic acid, eicosapentaenoic acid, isohexadecanoic acid, anteisoheneicosanic acid, long-chain branched fatty acids, dimer acids and hydrogenated dimer acids.

Examples of the fluorine oil include perfluorodecane, perfluorooctane, and perfluoro polyether.

Examples of the (poly)alcohol include ethanol, isopropanol, glycerin, propylene glycol, 1,3-butylene glycol, and polyethylene glycol.

Examples of the other base include, but are not particularly limited to, those used for patches (plasters, tapes (reservoir-type, matrix-type, etc.) such as emplastrums, cataplasms, transdermal patches, microneedles, etc.), ointments, liquids for external use (liniments, lotions, etc.), sprays (aerosols for external use, pump sprays, etc.) creams, gels, eye drops, eye ointments, nasal drops, suppositories, semisolids for application to rectum, and enema agents.

2.2 Other Additive Component

The formulation of the present invention may contain other additive components depending on the dosage form and the purpose of use.

The additive component is not particularly limited and examples thereof include excipients, coloring agents, lubricants, binders, emulsifiers, thickening agents, wetting agents, stabilizers, preservatives, solvents, solubilizers, suspending agents, buffering agents, pH adjusting agents, gels, adhesives, antioxidants, absorption enhancers, irritation reducing agents, antiseptics, chelating agents, and dispersing agents.

Also, the formulation of the present invention allows the particle without the base phase contained or the particle-containing base phase when contained (hereinafter these are generically referred to as “particle-containing basic component”) to be further dispersed in another component. In that case, the formulation of the present invention is provided by mixing and dispersing or emulsifying the particle or the particle-containing basic component into a component that incompletely dissolves the particle or the particle-containing basic component. The selection may be appropriately performed corresponding to the dosage form without specific limitations. For example, in order to provide patches (plasters, tapes (reservoir-type, matrix-type, etc.) such as emplastrums, cataplasms, transdermal patches, microneedles, etc.), ointments, liquids for external use (liniments, lotions, etc.), sprays (aerosols for external use, pump sprays, etc.), creams, gels, eye drops, eye ointments, nasal drops, suppositories, semisolids for application to rectum, and enema agents, the particle or the particle-containing basic component may be mixed and dispersed or emulsified into the base for use in each dosage form.

In particular, the particle of the present invention is stable in a process at 60° C. or higher for manufacturing the formulation. When the particle is used in patches or the like of which the formulation include a manufacturing process at 60° C. or higher, the stability in manufacturing at 60° C. or higher can be therefore further enhanced.

3. Method for Manufacturing a Particle and Formulation

The particle of the present invention can be manufactured, for example, by a method comprising the step of drying a W/O emulsion that contains an active ingredient and the water-soluble polymer of the present invention in the water phase, though not particularly limited thereto.

A W/O emulsion containing an active ingredient and the water-soluble polymer of the present invention in the aqueous phase, may be obtained, for example, by mixing an aqueous solvent (e.g., water, buffer aqueous solution, etc.) containing the active ingredient and the water-soluble polymer with an oily solvent (e.g. cyclohexane, hexane, toluene, etc.) containing a surfactant. The aqueous solvent containing an active ingredient may contain additive components such as absorption enhancers, irritation reducing agents, etc., in addition to the active ingredient on an as needed basis. The oily solvent containing a surfactant may contain additive components such as irritation reducing agents, analgesics, absorption enhancers, stabilizers, etc., in addition to the active ingredient on an as needed basis. The mixing method is not particularly limited as long as a W/O emulsion can be formed, and examples of the method include stirring by a homogenizer or the like. The stirring by a homogenizer may be performed under conditions, for example, at about 5000 to 50000 rpm, more preferably about 10000 to 30000 rpm.

The weight ratio between the active ingredient and the surfactant (active ingredient weight/surfactant weight) in the W/O emulsion is, for example, 1:2 to 1:100, preferably 1:3 to 1:50, still more preferably 1:3 to 1:30.

The weight ratio between the active ingredient and the water-soluble polymer of the present invention in the W/O emulsion (weight of active ingredient:weight of water-soluble polymer of the present invention) is, for example, 1:0.01 to 1:10, preferably 1:0.02 to 1:5, more preferably 1:0.05 to 1:2, still more preferably 1:0.08 to 1:1.5.

The method for drying a W/O emulsion is not particularly limited as long as the solvents (aqueous solvent and oily solvent) in the emulsion can be removed, and examples thereof include freeze drying and vacuum drying, preferably freeze drying. The formation of the particle can be confirmed through particle size measurement and with use of an optical microscope after drying and dispersing in a base such as isopropyl myristate on an as needed basis.

The particle of the present invention may be used as it is, or may be dispersed in the base or the like for use.

Further, from the particle of the present invention, a formulation can be manufactured, for example, by solution coating. In solution coating, in addition to the particle of the present invention and a base, optional additive components such as an absorption enhancer, a thickener and a gelling agent are added to a solvent such as hexane, toluene or ethyl acetate at a predetermined ratio, and stirred to prepare a homogeneous solution. The solid concentration in the solution is preferably 10 to 80 wt %, more preferably 20 to 60 wt %.

Subsequently, the solution that contains each of the components is uniformly applied onto a release liner (e.g., silicone treated polyester film) with an applicator such as a knife coater, a comma coater and a reverse coater, and dried for completion of a drug-containing layer, on which a substrate is laminated, so that a formulation can be obtained. Depending on the type of substrate, after formation of the layer on a substrate, a release layer may be laminated on the surface of the layer.

In an another method, for example, a base and additive components such as an absorption enhancer, a stabilizer, a thickener, and a gelling agent are added to the particle of the present invention on an as needed basis and mixed. Corresponding to application, the mixture may be held on a natural fabric member of gauze, cotton wool, or the like, on a synthetic fiber fabric member of polyester, polyethylene, or the like, on a woven or non-woven fabric made from an appropriate combination thereof, or on a permeable membrane by lamination, impregnation or the like, and further covered with an adhesive cover material or the like for use.

The formulation thus obtained is appropriately cut into a shape such as ellipse, a circle, a square, and a rectangle, depending on the intended use. An adhesive layer may be provided in the periphery on an as needed basis.

In another method, for example, an eye drop-type formulation can be manufactured. The eye drop liquid may be prepared by widely used techniques. Pharmaceutically acceptable additives may be added to the particle of the present invention on an as needed basis. The concentration of an active ingredient is 0.0001 to 5 wt %, preferably 0.0005 to 3 wt %, particularly preferably 0.001 to 1 wt %. The formulation liquid may be subjected to filter sterilization or other sterilization. Although the sterilization method is not particularly limited as long as the resulting formulation liquid can be sterilized, filtration sterilization preferably with use of a filtration sterilization filter having a pore size of 0.1 to 0.5 μm is preferred.

### EXAMPLES

The present invention is described in detail as follows with reference to Examples, though the present invention is not limited thereto.

### Example 1

### Active ingredient:Na hyaluronate:surfactant=1:0.1:30

In 40 g of pure water, 0.1 g of memantine hydrochloride (manufactured by Tokyo Chemical Industry Co., Ltd.) and 0.01 g of Na hyaluronate (manufactured by Kikkoman Biochemifa Co., molecular weight: 80000) were dissolved, to which a solution of 3.0 g of sucrose laurate (manufactured by Mitsubishi Chemical Foods Corporation, L-195; HLB value: 1) dissolved in 80 g of cyclohexane was added and stirred with a homogenizer (10000 rpm). The mixture was then freeze-dried for 2 days, so that a particle that contains an active ingredient, a surfactant, Na hyaluronate were obtained. In 850 mg of isotridecyl isononanate (manufactured by Kokyu Alcohol Kogyo Co., Ltd., KAK139, SP value: 8.2), 150 mg of the resulting particle was dispersed to manufacture a formulation.

### Example 2

### Active ingredient:Na hyaluronate:surfactant=1:0.1:30

A formulation was manufactured in the same manner as in Example 1, except that Na hyaluronate (manufactured by Kikkoman Biochemifa Co., molecular weight: 600000) was used instead of Na hyaluronate (manufactured by Kikkoman Biochemifa Co., molecular weight: 80000).

### Example 3

### Active ingredient:Na hyaluronate:surfactant=1:0.1:30

A formulation was manufactured in the same manner as in Example 1, except that Na hyaluronate (manufactured by Wako Pure Chemical Industries, Ltd., molecular weight: 1000000) was used instead of Na hyaluronate (manufactured by Kikkoman Biochemifa Co., molecular weight: 80000).

### Comparative Example 1

### Active ingredient:surfactant=1:30

A formulation was manufactured in the same manner as in Example 1, except that Na hyaluronate (manufactured by Kikkoman Biochemifa Co., molecular weight: 80000) was not added.

### Comparative Example 2

### Active ingredient: BSA: surfactant=1:0.1:30

A formulation was manufactured in the same manner as in Example 1, except that 0.01 g of BSA (manufactured by Sigma-Aldrich Corporation) was used instead of 0.01 g of Na hyaluronate (manufactured by Kikkoman Biochemifa Co., molecular weight: 80000).

### Comparative Example 3

### Active ingredient:BSA:surfactant=1:0.2:30

A formulation was manufactured in the same manner as in Example 1, except that 0.02 g of BSA (manufactured by Sigma-Aldrich Corporation) was used instead of 0.01 g of Na hyaluronate (manufactured by Kikkoman Biochemifa Co., molecular weight: 80000).

### Comparative Example 4

### Active ingredient:BSA:surfactant=1:0.5:30

A formulation was manufactured in the same manner as in Example 1, except that 0.05 g of BSA (manufactured by Sigma-Aldrich Corporation) was used instead of 0.01 g of Na hyaluronate (manufactured by Kikkoman Biochemifa Co., molecular weight: 80000).

### Comparative Example 5

### Active ingredient:BSA:surfactant=1:0.8:30

A formulation was manufactured in the same manner as in Example 1, except that 0.08 g of BSA (manufactured by Sigma-Aldrich Corporation) was used instead of 0.01 g of Na hyaluronate (manufactured by Kikkoman Biochemifa Co., molecular weight: 80000).

### Test Example 1

### Shape Stability Test 1

Formulations in Examples 1 to 3 and Comparative Examples 1 to 5 were stored at 40° C. After initiation of the storage, the state of the particle in the formulations was observed at fixed intervals with an optical microscope, so that the time period until change in the shape of the particle was measured. The shape stability is enhanced as the time period increases. The results are shown in Table 1.

TABLE 1

| | | Compo- | Time period | Cumulative |
|---|---|---|---|---|
| | | nent | until change | permeation |
| | | weight | in the shape | amount after |
| | | ratio | of the parti- | 48 hours |
| | Component C | (A:C:B) | cle (week) | (mg/cm²) |
| Example 1 | Na hyaluronate | 1:0.1:30 | 8 | 0.82 |
| | (molecular | | | |
| | weight: 80000) | | | |
| Example 2 | Na hyaluronate | | 8 | 0.85 |
| | (molecular | | | |
| | weight: 600000) | | | |
| Example 3 | Na hyaluronate | | 12 | 0.61 |
| | (molecular | | | |
| | weight: 1000000) | | | |
| Comparative | None | 1:0:30 | 1 | — |
| Example 1 | | | | |
| Comparative | BSA | 1:0.1:30 | 1 | — |
| Example 2 | | | | |
| Comparative | | 1:0.2:30 | 2 | 0.66 |
| Example 3 | | | | |
| Comparative | | 1:0.5:30 | 4 | 0.64 |
| Example 4 | | | | |
| Comparative | | 1:0.8:30 | 4 | 0.64 |
| Example 5 | | | | |

The particle obtained by adding Na hyaluronate as the component C (Examples 1 to 3) maintained the stable shape for a longer period in comparison with the particle without addition of the component C (Comparative Example 1). Further, the particle obtained by adding Na hyaluronate as the component C (Examples 1 to 3) maintained the stable shape for a longer period in comparison with the particle to which the same amount of BSA was added as the component C (Comparative Example 2).

### Test Example 2

### Hairless Rat Skin Permeation Test

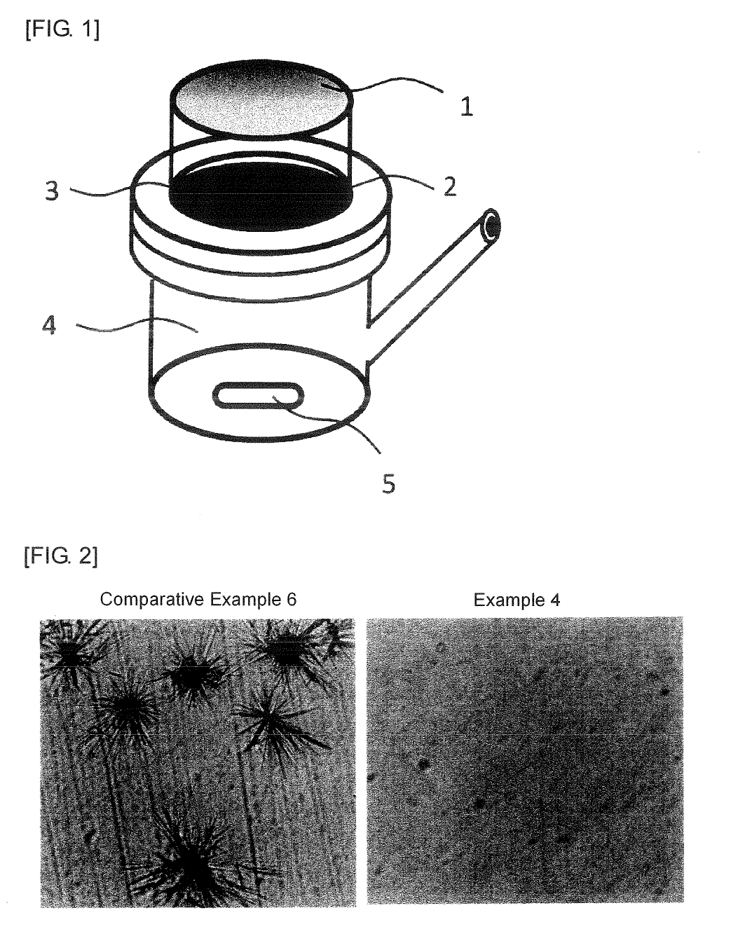

A hairless rat skin (manufactured by Japan SLC, Inc., excised from 8-week-old HWY/Slc) was set in a drug skin permeation test cell (FIG. 1). To the top of the device, 2 g (about 7.07 cm²) of each of the formulations in Examples 1 to 3 and Comparative Examples 3 to 5 was applied. A buffer solution containing 5×10⁻⁴ M of NaH₂PO₄, 2×10⁻⁴ M of Na₂HPO₄, 1.5×10⁻⁴ M of NaCl, and 10 ppm of gentamicin sulfate (manufactured by Wako Pure Chemical Industries, Ltd., G1658) in distilled water at a pH 7.2 adjusted with NaOH was put in the receptor layer at the bottom. The device was set in a thermostat chamber kept at 32° C. after initiation of the test. After 48 hours from the initiation of the test, 1 ml of the liquid was sampled from the receptor layer at the bottom in the thermostat chamber, and immediately after sampling, 1 ml of liquid having the same composition was supplemented. Methanol was added to each of the collected receptor liquid samples for extraction of eluted lipid and the like. After centrifugation, the memantine hydrochloride concentration in the supernatant was determined by gas chromatography (GC) (unit: manufactured by Shimadzu Corporation, GC-2014Plus; column for use: manufactured by JEOL Ltd., ZB-1, length: 30 m, inner diameter: 0.32 mm).

The calculated cumulative permeation amount (mg/cm²) after 48 hours is shown in Table 1. The permeabilities in Examples 1 to 3 are equivalent or higher than those in Comparative Examples 3 to 5.

### Example 4

In 40 g of pure water, 0.2 g of donepezil hydrochloride (manufactured by Kaneda Co., Ltd.) and 0.2 g of a phospholipid-like water-soluble polymer (manufactured by NOF Corporation, pharmaceutical excipient LIPIDURE-PMB, molecular weight: about 600000) were dissolved, to which a solution of 3.0 g of sucrose oleate (manufactured by Mitsubishi Chemical Foods Corporation, O-170; HLB value: 1) dissolved in 80 g of cyclohexane was added and stirred with a homogenizer (10000 rpm) The mixture was then freeze-dried for 2 days, so that a particle containing an active ingredient, a surfactant, and a phospholipid-like water-soluble polymer were obtained.

To 30 parts by weight of the particle obtained, 20 parts by weight of a styrene-isoprene-styrene block copolymer (SIS, manufactured by Zeon Corporation, QUINTAC 3520), 10 parts by weight of alicyclic saturated hydrocarbon resin (manufactured by Arakawa Chemical Industries, Ltd., ARKON P115), and 40 parts by weight of the liquid paraffin (manufactured by Wako Pure Chemical Industries, Ltd., density: 0.800 to 0.835 g/mL) were blended. To the mixture, cyclohexane was added to give a concentration of solid content of 30 wt %, and mixed until a uniform state was obtained. An adhesive layer solution was thus prepared.

Subsequently, silicone was applied to one surface of a release substrate made of polyethylene terephthalate film having a thickness of 38 μm to prepare a release-treated release sheet. The adhesive layer solution was applied to a release-treated surface of the release sheet, and dried at 60° C. for 30 minutes, so that a laminate having an adhesive layer on the release-treated surface of the release sheet was manufactured. Subsequently, a substrate made of polyethylene terephthalate film having a thickness of 38 μm was arranged. In producing of a tape, one surface of the substrate and the adhesive layer of the laminate were layered face to face, so that the adhesive layer of the laminate was transferred to the substrate to obtain a unified laminate.

### Comparative Example 6

A tape was manufactured in the same manner as in Example 4, except that the phospholipid-like water-soluble polymer was not added.

### Test Example 3

### Stability Test 2

After the tapes in Example 4 and Comparative Example 6 were stored at room temperature for 10 days, the surfaces of the tapes were observed with an optical microscope. The observed images are shown in FIG. 2.

As shown in FIG. 2, in the case with the particle to which no phospholipid-like water-soluble polymer was added (Comparative Example 6), crystal precipitation was observed. It is presumed that the precipitation was caused by leakage of the active ingredient due to change in the shape of the particle. In contrast, in the case with the particle to which a phospholipid-like water-soluble polymer was added (Example 4), no crystal precipitation was observed, which suggests that the shape of the particle is stable.

### Example 5

### Active ingredient:phospholipid-like water-soluble polymer:surfactant=1:0.5:15

In 40 g of pure water, 0.2 g of donepezil hydrochloride (manufactured by Kaneda Co., Ltd.) and 0.1 g of a phospholipid-like water-soluble polymer (manufactured by NOF Corporation, pharmaceutical excipient LIPIDURE-PMB, molecular weight: about 600000) were dissolved, to which a solution of 3.0 g of sucrose erucate (manufactured by Mitsubishi Chemical Foods Corporation, trade name “ER-290”; HLB value: 2) dissolved in 80 g of cyclohexane was added and stirred with a homogenizer (10000 rpm). The mixture was then freeze-dried for 2 days, so that a particle containing an active ingredient, a surfactant, and a phospholipid-like water-soluble polymer were obtained. In a mixed base of 674 mg of plastibase (manufactured by Taisho Pharmaceutical Co., Ltd., Japanese Pharmacopoeia) and 101 mg of isopropyl myristate (IPM, manufactured by Wako Pure Chemical Industries, Ltd.), 225 mg of the resulting particle was dispersed to manufacture a formulation.

### Example 6

### Active ingredient:phospholipid-like water-soluble polymer:surfactant=1:0.5:15

A formulation was manufactured in the same manner as in Example 5, except that 150 mg of the particle obtained in Example 5 was dispersed in a mixed base of 748 mg of plastibase (manufactured by Taisho Pharmaceutical Co., Ltd., Japanese Pharmacopoeia) and 102 mg of isopropyl myristate (IPM, manufactured by Wako Pure Chemical Industries, Ltd.).

### Example 7

### Active ingredient:phospholipid-like water-soluble polymer:surfactant=1:0.5:15

A formulation was manufactured in the same manner as in Example 5, except that 75 mg of the particle obtained in Example 5 was dispersed in a mixed base of 823 mg of plastibase (manufactured by Taisho Pharmaceutical Co., Ltd., Japanese Pharmacopoeia) and 102 mg of isopropyl myristate (IPM, manufactured by Wako Pure Chemical Industries, Ltd.).

### Comparative Example 7

### Active ingredient:surfactant=1:15

A formulation was manufactured in the same manner as in Example 5, except that no phospholipid-like water-soluble polymer was used.

### Comparative Example 8

### Active ingredient:surfactant=1:15

A formulation was manufactured in the same manner as in Example 6, except that no phospholipid-like water-soluble polymer was used.

### Comparative Example 9

### Active ingredient:surfactant=1:15

A formulation was manufactured in the same manner as in Example 7, except that no phospholipid-like water-soluble polymer was used.

According to the Test Example 1 (Shape stability test 1) and the Test Example 2 (Hairless rat skin permeability test), the formulations obtained in Examples 5 to 7 and Comparative Examples 7 to 9 were evaluated on the time period until change in the shape of the particle and the cumulative permeation amount after 48 hours. The results are shown in the following Table 2.

TABLE 2

| | | | | | | | | Cumulative |
|---|---|---|---|---|---|---|---|---|
| | | | | | | | Time period until change in | permeation |
| | Core | Shell | Additive | Ratio | Dispersion medium | Particle concentration | the shape of the particle | amount after 48 hours |
| Example 5 | DP | ER | Lipidure | 1:0.5:15 | Plastibase (13% IPM) | 22.5% | 3 months or more | 0.12 |
| Example 6 | DP | ER | Lipidure | 1:0.5:15 | Plastibase (12% IPM) | 15.0% | 3 months or more | 0.09 |
| Example 7 | DP | ER | Lipidure | 1:0.5:15 | Plastibase (11% IPM) | 7.5% | 3 months or more | 0.06 |
| Comparative | DP | ER | — | 1:15 | Plastibase (13% IPM) | 22.5% | 1 month | 0.12 |
| Example 7 | | | | | | | | |
| Comparative | DP | ER | — | 1:15 | Plastibase (12% IPM) | 15.0% | 1 month | 0.09 |
| Example 8 | | | | | | | | |
| Comparative | DP | ER | — | 1:15 | Plastibase (11% IPM) | 7.5% | 1 month | 0.06 |
| Example 9 | | | | | | | | |

As shown in Table 2, the particle obtained by adding the phospholipid-like water-soluble polymer (Examples 5 to 7) maintained the stable shape for a longer period in comparison with the particle without addition of phospholipid-like water-soluble polymer (Comparative Examples 7 to 9).

### Example 8

In 40 g of pure water, 0.2 g of donepezil hydrochloride (manufactured by Kaneda Co., Ltd.) and 0.02 g of Na hyaluronate (manufactured by Kikkoman Biochemifa Co., molecular weight: 600000) were dissolved, to which a solution of 3.0 g of sucrose erucate (manufactured by Mitsubishi Chemical Foods Corporation, trade name “ER-290”; HLB value: 2) dissolved in 80 g of cyclohexane was added and stirred with a homogenizer (10000 rpm). The mixture was then freeze-dried for 2 days, so that a particle containing an active ingredient, a surfactant, and a phospholipid-like water-soluble polymer were obtained.

In 30 parts by weight of the obtained particle, 30 parts by weight of a styrene-isoprene-styrene block copolymer (SIS, manufactured by Zeon Corporation, QUINTAC 3520), 20 parts by weight of alicyclic saturated hydrocarbon resin (manufactured by Arakawa Chemical Industries, Ltd., ARKON P100), and 20 parts by weight of a liquid paraffin (manufactured by Wako Pure Chemical Industries Ltd., density: 0.800 to 0.835 g/mL) were blended, to which cyclohexane was added to adjust the solid content to 30 wt %. The mixture was then mixed until uniformity is achieved, so that an adhesive layer solution was prepared.

Silicone was then applied to a surface of a release substrate made of polyethylene terephthalate film having a thickness of 38 μm, so that a release sheet with a release treatment was prepared. The adhesive layer solution was applied to the release-treated surface of the release sheet and dried at 60° C. for 30 minutes, so that a laminate including an adhesive layer formed on the release-treated surface of the release sheet was made. A support made of polyethylene terephthalate film having a thickness of 38 μm was then prepared. One face of the support and the adhesive layer of the laminate were superimposed to face each other, so that a tape was manufactured through laminate integration by transferring the adhesive layer of the laminate to the support.

### Example 9

A tape was manufactured in the same manner as in Example 8, except that Na hyaluronate (manufactured by Wako Pure Chemical Industries, Ltd., molecular weight: 1000000) was used instead of the Na hyaluronate in Example 8.

### Example 10

A tape was manufactured in the same manner as in Example 8, except that phospholipid-like water-soluble polymer (manufactured by NOF Corporation, pharmaceutical excipient LIPIDURE-PMB, molecular weight: about 600000) was used instead of the Na hyaluronate in Example 8.

### Example 11

In 40 g of pure water, 0.2 g of donepezil hydrochloride (manufactured by Kaneda Co., Ltd.) and 0.2 g of a phospholipid-like water-soluble polymer (manufactured by NOF Corporation, pharmaceutical excipient LIPIDURE-PMB, molecular weight: about 600000) were dissolved, to which a solution of 3.0 g of sucrose erucate (manufactured by Mitsubishi Chemical Foods Corporation, trade name “ER-290”; HLB value: 2) dissolved in 80 g of cyclohexane was added and stirred with a homogenizer (10000 rpm). The mixture was then freeze-dried for 2 days, so that a particle containing an active ingredient, a surfactant, and a phospholipid-like water-soluble polymer were obtained.

In 30 parts by weight of the obtained particle, 25.7 parts by weight of a styrene-isoprene-styrene block copolymer (SIS, manufactured by Zeon Corporation, QUINTAC 3520), 17.15 parts by weight of alicyclic saturated hydrocarbon resin (manufactured by Arakawa Chemical Industries, Ltd., ARKON P100), 17.15 parts by weight of a liquid paraffin (manufactured by Wako Pure Chemical Industries Ltd., density: 0.800 to 0.835 g/mL), and 10 parts by weight of isopropyl myristate (IPM, manufactured by Wako Pure Chemical Industries, Ltd.) were blended, to which cyclohexane was added to adjust the solid content to 30 wt %. The mixture was then mixed until uniformity is achieved, so that an adhesive layer solution was prepared. A tape was manufactured in the same manner as in Example 8, except that the adhesive layer solution thus prepared was used.

### Example 12

In 40 g of pure water, 0.2 g of donepezil hydrochloride (manufactured by Kaneda Co., Ltd.) and 0.02 g of a phospholipid-like water-soluble polymer (manufactured by NOF Corporation, pharmaceutical excipient LIPIDURE-PMB, molecular weight: about 600000) were dissolved, to which a solution of 3.0 g of sucrose erucate (manufactured by Mitsubishi Chemical Foods Corporation, trade name “ER-290”; HLB value: 2) dissolved in 80 g of cyclohexane was added and stirred with a homogenizer (10000 rpm). The mixture was then freeze-dried for 2 days, so that a particle containing an active ingredient, a surfactant, and a phospholipid-like water-soluble polymer were obtained. A tape was manufactured in the same manner as in Example 11, except that the particle thus obtained was used.

### Example 13

A tape was manufactured in the same manner as in Example 11, except that sucrose oleate (manufactured by Mitsubishi Chemical Foods Corporation, O-170; HLB value: 1) was used instead of sucrose erucate.

### Example 14

A tape was manufactured in the same manner as in Example 12, except that sucrose oleate (manufactured by Mitsubishi Chemical Foods Corporation, O-170; HLB value: 1) was used instead of sucrose erucate.

### Example 15

A tape was manufactured in the same manner as in Example 11, except that castor oil (manufactured by Kosakai Pharmaceutical Co., Ltd.) was used instead of liquid paraffin (manufactured by Wako Pure Chemical Industries Ltd., density: 0.800 to 0.835 g/mL).

### Example 16

A tape was manufactured in the same manner as in Example 12, except that castor oil (manufactured by Kosakai Pharmaceutical Co., Ltd.) was used instead of liquid paraffin (manufactured by Wako Pure Chemical Industries Ltd., density: 0.800 to 0.835 g/mL).

### Example 17

A tape was manufactured in the same manner as in Example 13, except that castor oil (manufactured by Kosakai Pharmaceutical Co., Ltd.) was used instead of liquid paraffin (manufactured by Wako Pure Chemical Industries Ltd., density: 0.800 to 0.835 g/mL).

### Example 18

A tape was manufactured in the same manner as in Example 14, except that castor oil (manufactured by Kosakai Pharmaceutical Co., Ltd.) was used instead of liquid paraffin (manufactured by Wako Pure Chemical Industries Ltd., density: 0.800 to 0.835 g/mL).

### Example 19

In 40 g of pure water, 0.2 g of donepezil hydrochloride (manufactured by Kaneda Co., Ltd.) and 0.2 g of a phospholipid-like water-soluble polymer (manufactured by NOF Corporation, pharmaceutical excipient LIPIDURE-PMB, molecular weight: about 600000) were dissolved, to which a solution of 2.0 g of sucrose erucate (manufactured by Mitsubishi Chemical Foods Corporation, trade name “ER-290”; HLB value: 2) dissolved in 80 g of cyclohexane was added and stirred with a homogenizer (10000 rpm). The mixture was then freeze-dried for 2 days, so that a particle containing an active ingredient, a surfactant, and a phospholipid-like water-soluble polymer were obtained. A tape was manufactured in the same manner as in Example 13, except that the particle thus obtained were used.

### Example 20

A tape was manufactured in the same manner as in Example 19, except that a phospholipid-like water-soluble polymer (manufactured by NOF Corporation, pharmaceutical excipient LIPIDURE-HM, molecular weight: about 100000) was used instead of the phospholipid-like water-soluble polymer (manufactured by NOF Corporation, pharmaceutical excipient LIPIDURE-PMB, molecular weight: about 600000).

### Example 21

A tape was manufactured in the same manner as in Example 19, except that a phospholipid-like water-soluble polymer (manufactured by NOF Corporation, pharmaceutical excipient LIPIDURE-BL206, molecular weight: about 300000) was used instead of the phospholipid-like water-soluble polymer (manufactured by NOF Corporation, pharmaceutical excipient LIPIDURE-PMB, molecular weight: about 600000).

### Example 22

A tape was manufactured in the same manner as in Example 19, except that a phospholipid-like water-soluble polymer (manufactured by NOF Corporation, pharmaceutical excipient LIPIDURE-BL1201, molecular weight: about 400000) was used instead of the phospholipid-like water-soluble polymer (manufactured by NOF Corporation, pharmaceutical excipient LIPIDURE-PMB,. molecular weight: about 600000).

### Comparative Example 10

A tape was manufactured in the same manner as in Example 10, except that a particle was obtained without use of a phospholipid-like water-soluble polymer.

### Comparative Example 11

A tape was manufactured in the same manner as in Example 11, except that a particle was obtained without use of a phospholipid-like water-soluble polymer.

### Comparative Example 12

A tape was manufactured in the same manner as in Example 13, except that a particle was obtained without use of a phospholipid-like water-soluble polymer.

### Comparative Example 13

A tape was manufactured in the same manner as in Example 16, except that a particle was obtained without use of a phospholipid-like water-soluble polymer.

### Comparative Example 14

A tape was manufactured in the same manner as in Example 17, except that a particle was obtained without use of a phospholipid-like water-soluble polymer.

### Comparative Example 15

A tape was manufactured in the same manner as in Example 19, except that a particle was obtained without use of a phospholipid-like water-soluble polymer.

According to the Test Example 1 (Shape stability test 1) and the Test Example 2 (Hairless rat skin permeability test), the tapes (formulations) obtained in Examples 8 to 22 and Comparative Examples 10 to 15 were evaluated on the time period until change in the shape of the particle and the cumulative permeation amount after 48 hours. The results are shown in the following Table 3.

TABLE 3

| | | | | | | | | | | | | | Cumulative |
|---|---|---|---|---|---|---|---|---|---|---|---|---|---|
| | | | | | | | | | | | | Time period | permeation |
| | | | | | Tape | | | | | | | until change | amount after |
| | | | | | Par- | | | | | | | in the shape | 48 hours |
| | Core | Shell | Additive | Ratio | ticle | SIS | Tackifier | Plasticizer | | Plasticizer | | of the particle | (mg/cm²) |
| Example 8 | DP | ER | HA | 1:0.1:15 | 30 | 30 | 20 | Liquid paraffin | 20 | | | 1 month | 0.013 |
| | | | (Mw: 600000) | | | | | | | | | | |
| Example 9 | DP | ER | HA | 1:0.1:15 | 30 | 30 | 20 | Liquid paraffin | 20 | | | 1 month | 0.014 |
| | | | (Mw: 1000000) | | | | | | | | | | |
| Example 10 | DP | ER | Lipidure-PMB | 1:0.1:15 | 30 | 30 | 20 | Liquid paraffin | 20 | | | 3 months or more | 0.020 |
| Example 11 | DP | ER | Lipidure-PMB | 1:1:15 | 30 | 25.7 | 17.15 | Liquid paraffin | 17.15 | IPM | 10 | 3 months or more | 0.037 |
| Example 12 | DP | ER | Lipidure-PMB | 1:0.1:15 | 30 | 25.7 | 17.15 | Liquid paraffin | 17.15 | IPM | 10 | 3 months or more | 0.036 |
| Example 13 | DP | O | Lipidure-PMB | 1:1:15 | 30 | 25.7 | 17.15 | Liquid paraffin | 17.15 | IPM | 10 | 3 months or more | 0.058 |
| Example 14 | DP | O | Lipidure-PMB | 1:0.1:15 | 30 | 25.7 | 17.15 | Liquid paraffin | 17.15 | IPM | 10 | 3 months or more | 0.057 |
| Example 15 | DP | ER | Lipidure-PMB | 1:1:15 | 30 | 25.7 | 17.15 | Castor oil | 17.15 | IPM | 10 | 3 months or more | 0.066 |
| Example 16 | DP | ER | Lipidure-PMB | 1:0.1:15 | 30 | 25.7 | 17.15 | Castor oil | 17.15 | IPM | 10 | 3 months or more | 0.061 |
| Example 17 | DP | O | Lipidure-PMB | 1:1:15 | 30 | 25.7 | 17.15 | Castor oil | 17.15 | IPM | 10 | 3 months or more | 0.061 |
| Example 18 | DP | O | Lipidure-PMB | 1:0.1:15 | 30 | 25.7 | 17.15 | Castor oil | 17.15 | IPM | 10 | 3 months or more | 0.075 |
| Example 19 | DP | O | Lipidure-PMB | 1:1:10 | 30 | 25.7 | 17.15 | Liquid paraffin | 17.15 | IPM | 10 | 3 months or more | 0.052 |
| Example 20 | DP | O | Lipidure-HM | 1:1:10 | 30 | 25.7 | 17.15 | Liquid paraffin | 17.15 | IPM | 10 | 3 months or more | 0.051 |
| Example 21 | DP | O | Lipidure- | 1:1:10 | 30 | 25.7 | 17.15 | Liquid paraffin | 17.15 | IPM | 10 | 3 months or more | 0.051 |
| | | | BL206 | | | | | | | | | | |
| Example 22 | DP | O | Lipidure- | 1:1:10 | 30 | 25.7 | 17.15 | Liquid paraffin | 17.15 | IPM | 10 | 3 months or more | 0.059 |
| | | | BL1201 | | | | | | | | | | |
| Comparative | DP | ER | — | 1:15 | 30 | 30 | 20 | Liquid paraffin | 20 | | | 2 weeks | 0.022 |
| Example 10 | | | | | | | | | | | | | |
| Comparative | DP | ER | — | 1:15 | 30 | 25.7 | 17.15 | Liquid paraffin | 17.15 | IPM | 10 | 2 weeks | 0.036 |
| Example 11 | | | | | | | | | | | | | |
| Comparative | DP | O | — | 1:15 | 30 | 25.7 | 17.15 | Liquid paraffin | 17.15 | IPM | 10 | 3 days | 0.045 |
| Example 12 | | | | | | | | | | | | | |
| Comparative | DP | ER | — | 1:15 | 30 | 25.7 | 17.15 | Castor oil | 17.15 | IPM | 10 | 2 weeks | 0.075 |
| Example 13 | | | | | | | | | | | | | |
| Comparative | DP | O | — | 1:15 | 30 | 25.7 | 17.15 | Castor oil | 17.15 | IPM | 10 | 3 days | 0.075 |
| Example 14 | | | | | | | | | | | | | |
| Comparative | DP | O | — | 1:10 | 30 | 25.7 | 17.15 | Liquid paraffin | 17.15 | IPM | 10 | 3 days | 0.048 |
| Example 15 | | | | | | | | | | | | | |

As shown in Table 3, the particle obtained by adding Na hyaluronate or a phospholipid-like water-soluble polymer (Examples 8 to 22) maintained the stable shape for a longer period in comparison with the particle without addition of Na hyaluronate or a phospholipid-like water-soluble polymer (Comparative Examples 10 to 15).

Also, as shown in Tables 2 and 3, in Examples 5 to 7 and Examples 10 to 22 with use of the particle obtained by adding a phospholipid-like water-soluble polymer, the stable shape was maintained for a further longer period in comparison with those without addition of a phospholipid-like water-soluble polymer, in the case with the same active ingredient for use, though the obtained data differed depending on the type of the active ingredient.

### REFERENCE SIGNS LIST

1 PARAFILM
2 SKIN
3 FORMULATION
4 RECEPTOR LIQUID (pH=7.2, PHOSPHATE BUFFER SOLUTION)
5 STIRRING BAR

1. A particle comprising:
a first fraction containing an active ingredient,
a second fraction containing a surfactant: and
a water-soluble polymer having 2-methacryloyloxyethyl phosphorylcholine as a constituent unit.

2. The particle according to claim 1, wherein the particle includes a part or the whole of the surface of the first fraction directly or indirectly covered with the second fraction.

3. The particle according to claim 1, wherein the first fraction contains the water-soluble polymer.

4. The particle according to claim 1, having a water content of 20 wt % or less.

5. The particle according to claim 1, wherein the water-soluble polymer has a molecular weight of 2000 or more.

6. The particle according to claim 1, wherein the water-soluble polymer has a molecular weight of 50000 or more.

7. (canceled)

8. The particle according to claim 1, wherein the water-soluble polymer having 2-methacryloyloxyethyl phosphorylcholine as a constituent unit is a copolymer of 2-methacryloyloxyethyl phosphorylcholine with a hydrophobic monomer.

9. The particle according to claim 1, wherein a weight ratio between the active ingredient and the water-soluble polymer is 1:0.02 to 1:5.

10. A formulation comprising the particle according to claim 1.

11. The formulation according to claim 10, having a water content of 20 wt % or less.

12. The formulation according to claim 10, wherein a weight ratio between the active ingredient and the surfactant is 1:3 to 1:50.

13. An external preparation comprising the formulation according to claim 10.

14. A cosmetic product comprising the formulation according to claim 10.
